SEQUENCE LISTING (68 sequences)

SEQ ID NO: 1 - DNA, 20 bp, Artificial Sequence
  CD19 F primer
cctggggtcccagtcctatg

SEQ ID NO: 2 - DNA, 20 bp, Artificial Sequence
  CD19 R primer
gctccagaggttggcatcat

SEQ ID NO: 3 - DNA, 20 bp, Artificial Sequence
  CD45 F primer
tcagtggtcccattgttgtg

SEQ ID NO: 4 - DNA, 20 bp, Artificial Sequence
  CD45 R primer
gcatctctgtggccttagct

SEQ ID NO: 5 - DNA, 17 bp, Artificial Sequence
  CD29 F primer
gccgcgcggaaaagatg

SEQ ID NO: 6 - DNA, 20 bp, Artificial Sequence
  CD29 R primer
acatcgtgcagaagtaggca

SEQ ID NO: 7 - DNA, 20 bp, Artificial Sequence
  CD73 F primer
tatccggtcgcccattgatg

SEQ ID NO: 8 - DNA, 20 bp, Artificial Sequence
  CD73 R primer
acgctatgctcaaaggcctt

SEQ ID NO: 9 - DNA, 20 bp, Artificial Sequence
  CD105 F primer
ccaagaccgggtctcaagac

SEQ ID NO: 10 - DNA, 20 bp, Artificial Sequence
  CD105 R primer
tgtaccagagtgcagcagtg

SEQ ID NO: 11 - DNA, 20 bp, Artificial Sequence
  CD166 F primer
gaacacgatgaggcagacga

SEQ ID NO: 12 - DNA, 23 bp, Artificial Sequence
  CD166 R primer
ccgaggtccttgtttacatgttt

SEQ ID NO: 13 - DNA, 21 bp, Artificial Sequence
  GAPDH F primer
aatcccatcaccatcttccag

SEQ ID NO: 14 - DNA, 18 bp, Artificial Sequence
  GAPDH R primer
atgacccttttggctccc

SEQ ID NO: 15 - DNA, 21 bp, Artificial Sequence
  NANOG F primer
tcctgaacctcagctacaaac

SEQ ID NO: 16 - DNA, 20 bp, Artificial Sequence
  NANOG R primer
gcgtcacaccattgctattc

SEQ ID NO: 17 - DNA, 20 bp, Artificial Sequence
  SOX2 F primer
catcacccacagcaaatgac

SEQ ID NO: 18 - DNA, 24 bp, Artificial Sequence
  SOX2 R primer
gaagtccaggatctctctcataaa

SEQ ID NO: 19 - DNA, 21 bp, Artificial Sequence
  REX1 F primer
gtttcgtgtgtccctttcaag

SEQ ID NO: 20 - DNA, 23 bp, Artificial Sequence
  REX1 R primer
ctgttatctgcttcatcctgttg

SEQ ID NO: 21 - DNA, 22 bp, Artificial Sequence
  ITGA6 F primer
cgaaaccaaggttctgagccca

SEQ ID NO: 22 - DNA, 22 bp, Artificial Sequence
  ITGA6 R primer
cttggatctccactgaggcagt

SEQ ID NO: 23 - DNA, 21 bp, Artificial Sequence
  CD146 F primer
gtgttgaatctgtcttgtgaa

SEQ ID NO: 24 - DNA, 16 bp, Artificial Sequence
  CD146 R primer
atgcctcagatcgatg

SEQ ID NO: 25 - DNA, 22 bp, Artificial Sequence
  TM4SF1 F primer
ggctactgtgtcattgtggcag

SEQ ID NO: 26 - DNA, 22 bp, Artificial Sequence
  TM4SF1 R primer
actcggaccatgtggaggtatc

SEQ ID NO: 27 - DNA, 21 bp, Artificial Sequence
  GAPDH F primer
aatcccatcaccatcttccag

SEQ ID NO: 28 - DNA, 21 bp, Artificial Sequence
  GAPDH R primer
cacgataccaaagttgtcatg

SEQ ID NO: 29 - DNA, 21 bp, Artificial Sequence
  GPX1 F primer
cagtcggtgtatgccttctcg

SEQ ID NO: 30 - DNA, 19 bp, Artificial Sequence
  GPX1 R primer
gagggacgccacattctcg

SEQ ID NO: 31 - DNA, 23 bp, Artificial Sequence
  GPX2 F primer
ggtagatttcaatacgttccggg

SEQ ID NO: 32 - DNA, 23 bp, Artificial Sequence
  GPX2 R primer
tgacagttctcctgatgtccaaa

SEQ ID NO: 33 - DNA, 19 bp, Artificial Sequence
  GPX3 F primer
agagccggggacaagagaa

SEQ ID NO: 34 - DNA, 21 bp, Artificial Sequence
  GPX3 R primer
atttgccagcatactgcttga

SEQ ID NO: 35 - DNA, 23 bp, Artificial Sequence
  GPX4 F primer
gaggcaagaccgaagtaaactac

SEQ ID NO: 36 - DNA, 20 bp, Artificial Sequence
  GPX4 R primer
ccgaactggttacacgggaa

SEQ ID NO: 37 - DNA, 20 bp, Artificial Sequence
  GPX7 F primer
cccaccactttaacgtgctc

SEQ ID NO: 38 - DNA, 21 bp, Artificial Sequence
  GPX7 R primer
ggcaaagctctcaatctcctt

SEQ ID NO: 39 - DNA, 23 bp, Artificial Sequence
  GSR F primer
ttccagaataccaacgtcaaagg

SEQ ID NO: 40 - DNA, 21 bp, Artificial Sequence
  GSR R primer
gttttcggccagcagctattg

SEQ ID NO: 41 - DNA, 21 bp, Artificial Sequence
  SOD1 F primer
ggtgggccaaaggatgaagag

SEQ ID NO: 42 - DNA, 20 bp, Artificial Sequence
  SOD1 R primer
ccacaagccaaacgacttcc

SEQ ID NO: 43 - DNA, 21 bp, Artificial Sequence
  SOD2 F primer
gctccggttttggggtatctg

SEQ ID NO: 44 - DNA, 20 bp, Artificial Sequence
  SOD2 R primer
gcgttgatgtgaggttccag

SEQ ID NO: 45 - DNA, 20 bp, Artificial Sequence
  SOD3 F primer
atgctggcgctactgtgttc

SEQ ID NO: 46 - DNA, 19 bp, Artificial Sequence
  SOD3 R primer
ctccgccgagtcagagttg

SEQ ID NO: 47 - DNA, 21 bp, Artificial Sequence
  Catalase F primer
tgttgctggagaatcgggttc

SEQ ID NO: 48 - DNA, 23 bp, Artificial Sequence
  Catalase R primer
tcccagttaccatcttctgtgta

SEQ ID NO: 49 - DNA, 22 bp, Artificial Sequence
  TMX1 F primer
agtatgtcagcactctttcagc

SEQ ID NO: 50 - DNA, 21 bp, Artificial Sequence
  TMX1 R primer
cacactggcaatccaaggtct

SEQ ID NO: 51 - DNA, 23 bp, Artificial Sequence
  TXNIP F primer
ggtctttaacgaccctgaaaagg

SEQ ID NO: 52 - DNA, 22 bp, Artificial Sequence
  TXNIP R primer
acacgagtaacttcacacacct

SEQ ID NO: 53 - DNA, 21 bp, Artificial Sequence
  TXN F primer
gtgaagcagatcgagagcaag

SEQ ID NO: 54 - DNA, 23 bp, Artificial Sequence
  TXN R primer
cgtggctgagaagtcaactacta

SEQ ID NO: 55 - DNA, 22 bp, Artificial Sequence
  Col1A1 F primer
gattccctggacctaaaggtgc

SEQ ID NO: 56 - DNA, 22 bp, Artificial Sequence
  Col1A1 R primer
agcctctccatctttgccagca

SEQ ID NO: 57 - DNA, 23 bp, Artificial Sequence
  MCP-1 F primer
agaatcaccagcagcaagtgtcc

SEQ ID NO: 58 - DNA, 22 bp, Artificial Sequence
  MCP-1 R primer
tcctgaacccacttctgcttgg

SEQ ID NO: 59 - DNA, 22 bp, Artificial Sequence
  MCP-3 F primer
acagaaggaccaccagtagcca

SEQ ID NO: 60 - DNA, 23 bp, Artificial Sequence
  MCP-3 R primer
ggtgcttcataaagtcctggacc

SEQ ID NO: 61 - DNA, 22 bp, Artificial Sequence
  CCL5 F primer
cctgctgctttgcctacattgc

SEQ ID NO: 62 - DNA, 22 bp, Artificial Sequence
  CCL5 R primer
acacacttggcggttctttcgg

SEQ ID NO: 63 - DNA, 22 bp, Artificial Sequence
  PAI-1 F primer
ctcatcagccactggaaaggca

SEQ ID NO: 64 - DNA, 23 bp, Artificial Sequence
  PAI-1 R primer
gactcgtgaagtcagcctgaaac

SEQ ID NO: 65 - DNA, 22 bp, Artificial Sequence
  TGF-beta1 F primer
tacctgaacccgtgttgctctc

SEQ ID NO: 66 - DNA, 22 bp, Artificial Sequence
  TGF-beta1 R primer
gttgctgaggtatcgccaggaa

SEQ ID NO: 67 - DNA, 23 bp, Artificial Sequence
  TGF-beta3 F primer
ctaagcggaatgagcagaggatc

SEQ ID NO: 68 - DNA, 22 bp, Artificial Sequence
  TGF-beta3 R primer
tctcaacagccactcacgcaca

(19) United States
(12) Patent Application Publication
(10) Pub. No.: US 2022/0347225 A1
(43) Pub. Date: Nov. 3, 2022

(54) COMPOSITION COMPRISING SKELETAL MUSCLE STEM CELL-DERIVED EXOSOME AS ACTIVE INGREDIENT FOR IMPROVING SKIN CONDITION

(71) Applicants: Konkuk University Industrial Cooperation Corp., Seoul (KR); Konkuk University Glocal Industry-Academic Collaboration Foundation, Chungcheongbuk-do (KR)

(72) Inventors: Ssang-Goo CHO, Seoul (KR); Hyun Jin SHIN, Seoul (KR); Kyung Min LIM, Gwangju (KR); Min Chan GIL, Seoul (KR); Ahmed ABDEL DAYEM, Seoul (KR)

(73) Assignees: Konkuk University Industrial Cooperation Corp., Seoul (KR); Konkuk University Glocal Industry-Academic Collaboration Foundation, Chungcheongbuk-do (KR)

(21) Appl. No.: 17/633,264

(22) PCT Filed: Aug. 7, 2020
(86) PCT No.: PCT/KR2020/010496

§ 371 Date: Jun. 23, 2022

(30) Foreign Application Priority Data
Aug. 7, 2019 (KR) 10-2019-0095934

(51) Int. Cl. A61K 35/34; A61K 8/98; A61P 17/00; A61Q 19/00

(52) U.S. Cl. CPC A61K 35/34; A61K 8/98; A61P 17/00; A61Q 19/00

(57) ABSTRACT

The present disclosure relates to a composition containing extracellular vesicles isolated from skeletal muscle-derived stem cells as active ingredients, which exhibits the effect of skin aging delay, wound healing, scar reduction and skin whitening, thereby improving skin condition. The present disclosure can not only utilize tissues wasted after surgical operation, thus allowing easy supply of raw materials, but also takes advantage of natural substances, thereby being free from the risk of side effects even upon long-term administration. The composition of the present disclosure promotes the secretion of collagen, reduces reactive oxygen species and promotes re-epithelialization at wound site while remarkably inhibiting melanocyte proliferation, tyrosinase activity and melanogenesis. Accordingly, it can facilitate effective recovery from skin tissue damage caused by oxidative stress, physical wound, intrinsic aging at cell or tissue levels, or hyperpigmentation.

### TECHNICAL FIELD

The present disclosure relates to a composition for improving skin condition including skin whitening, which includes stem cell-derived exosomes isolated from skeletal muscle, specifically from orbicularis oculi muscle, as active ingredients.

### BACKGROUND ART

Orbicularis oculi muscle-derived stem cells (ORM-SCs) have self-renewal, proliferation and multi-lineage differentiation functions [1]. The stem cells located at the junctions of most organs are responsible for the maintenance and recovery of the organs throughout life [2]. According to recent researches, it was reported that the stem cells abundant in the ORM can differentiate into various types (fat, cartilage and bone cells) [3-5].

Ideal eyelid reconstruction should give natural appearance [6]. At present, upper eyelid plastic surgery is the most commonly performed cosmetic ophthalmic surgery in Korea. During the surgery, some of ORM tissues are removed and wasted. Recently, a few surgeons used local flaps instead of skin grafting or Z-plasty for treatment of lagophthalmos. The ORM tissue is used to treat the facial skin defects in the cheek, nose or lower eyelids [7, 8]. For facial burns, the ORM tissue plays an important role in transplantation and recovery [9].

ORM is a very specialized skeletal muscle and can be a good stem cell source owing to active vascularization and excellent accessibility [10]. In general, a sufficient number of ORM-SCs for invasive biopsy processes cannot be obtained because the amount of muscular tissues is limited.

The effect of these cells on tissue regeneration and recovery mostly derives from the factors (secretomes) secreted by them [11]. SC-derived extracellular vesicles (EVs) function usefully as mediators of skin regeneration and reconstruction [12].

Stromal cells and epithelial melanocytes play an important role in regulation of skin pigmentation [13].

The inventors of the present disclosure have isolated ORM-SCs having self-renewal, proliferation and multi-lineage differentiation abilities from orbicularis oculi muscle and have identified that EVs isolated from the ORM-SCs have an important role in regulating pigmentation and inhibiting melanin synthesis.

Throughout the present specification, many literatures and patent documents are cited. The disclosures of the cited literatures and patent documents are incorporated herein by reference in their entirety to describe the level of the art to which the present disclosure belongs and the contents of the present disclosure more clearly.

### DISCLOSURE

### Technical Problem

The inventors of the present disclosure have made consistent efforts to develop skin whitening materials derived from natural products, which have various skin-improving effects such as inhibition of pigmentation, skin tissue regeneration, prevention of aging, etc. without side effects and can be obtained easily. As a result, they have found out that extracellular vesicles secreted by stem cells isolated from skeletal muscle, particularly orbicularis oculi muscle tissue, which have been discarded as byproducts of upper eyelid plastic surgery, not only significantly inhibit melanocyte proliferation, tyrosinase activity and melanogenesis but also induce skin regeneration, wound healing, scar reduction and skin whitening by facilitating collagen secretion, reducing reactive oxygen species and promoting re-epithelialization at wound site, thereby delaying aging, and have completed the present disclosure.

The present disclosure is directed to providing a cosmetic composition for improving skin condition through antioxidation or skin whitening.

The present disclosure is also directed to providing a pharmaceutical composition for preventing or treating a hyperpigmentation disease.

Other purposes and advantages of the present disclosure will become more apparent by the detailed description, claims and drawings of the present disclosure.

### Technical Solution

In an aspect, the present disclosure provides a cosmetic composition for antioxidation or improvement of skin condition, which contains extracellular vesicles isolated from skeletal muscle-derived stem cells as active ingredients, wherein the improvement of skin condition is selected from a group consisting of skin aging delay, wound healing, scar reduction and skin whitening.

The inventors of the present disclosure have made consistent efforts to develop skin whitening materials derived from natural products, which have various skin-improving effects such as inhibition of pigmentation, skin tissue regeneration, prevention of aging, etc. without side effects and can be obtained easily. As a result, they have found out that extracellular vesicles secreted by stem cells isolated from skeletal muscle, particularly orbicularis oculi muscle tissue, which have been discarded as byproducts of upper eyelid plastic surgery, not only significantly inhibit melanocyte proliferation, tyrosinase activity and melanogenesis but also delay aging caused by oxidative stress by promoting collagen secretion, reducing reactive oxygen species and facilitating re-epithelialization at wound site and induce scar reduction and skin whitening, and thus can be used for a composition for improving skin condition.

In the present specification, the term “stem cell” refers to a cell capable of differentiating into various cell types constituting biological tissues. It refers to a multipotent or pluripotent cell that can regenerate unlimitedly to form specialized cells of tissues and organs. Skeletal muscle-derived stem cells can be obtained by selectively isolating the cells having stemness from a population of inhomogeneous cells obtained form skeletal muscle tissue or a culture thereof according to a common method.

In the present specification, the term “skeletal muscle” refers to a striated muscle tissue which is attached to a bone tissue through a tendon composed of collagen fibers and is controlled voluntarily by the somatic nervous system. The skeletal muscle that may be used in the present disclosure is specifically facial skeletal muscle, more specifically a skeletal muscle around eyes selected from a group consisting of levator palpebrae superioris muscle, corrugator supercilii muscle, depressor supercilii muscle and orbicularis oculi muscle.

Most specifically, the skeletal muscle used in the present disclosure is orbicularis oculi muscle.

In the present specification, the term “isolation” includes not only a process of selectively obtaining a desired substance (e.g., a stem cell or an extracellular vesicle) from a biological sample (e.g., a tissue or a cell culture) (positive isolation) but also a process of selectively removing impurities other than the desired substance (negative isolation). Accordingly, the term “isolation” is used with the same meaning as “obtainment”, “extraction” or “purification”. In the present disclosure, a process of isolating a stem cell from a skeletal muscle tissue includes, for example, separation of an antibody specific for a surface antigen of a target cell (e.g., FACS) separation based on the difference in the specific gravity of a target cell (e.g., phase separation or centrifugation). However, any method commonly used in the art for isolating a specific cell from an inhomogeneous sample may be used without being limited thereto.

In addition, the process of isolating extracellular vesicles from the stem cells includes separation of components in a cell culture based on the difference in specific gravity (e.g., ultrafiltration), separation based on size (e.g., ultracentrifugation or vacuum filtration) and separation based on affinity to a specific substrate (e.g., affinity chromatography). However, any method commonly used in the art for a target substance from an inhomogeneous sample based on the intrinsic physical properties of a target substance may be used without being limited thereto.

In the present specification, the term “extracellular vesicle” refers to a vesicle with a lipid bilayer structure secreted naturally by various cells, which includes specific molecules possessed by a cell from which it is derived, such as a protein, a nucleic acid, a lipid, a carbohydrate, etc. and is released to the extracellular environment through fusion of a multivesicular body with the plasma membrane. The extracellular vesicle has various diameters in a range of approximately 30-1,000 nm. In particular, an exosome is a small membrane vesicle with the smallest size of 50-200 nm.

In a specific exemplary embodiment of the present disclosure, the extracellular vesicle used in the present disclosure has a diameter of 50-200 nm, i.e., an exosome. More specifically, it has a diameter of 70-180 nm. Further more specifically, it has a diameter of 90-160 nm. Even more specifically, it has a diameter of 100-140 nm. Most specifically, it has a diameter of 100-120 nm.

In a specific exemplary embodiment of the present disclosure, the extracellular vesicle is contained in the composition of the present disclosure at a concentration of 5-100 μg/mL, more specifically 10-100 μg/mL, further more specifically 30-100 μg/mL, most specifically 50-100 μg/mL.

In a specific exemplary embodiment of the present disclosure, the stem cell of the present disclosure is positive for three or markers, more specifically four or markers, further more specifically five or markers, most specifically all the six markers, selected from a group consisting of CD105, CD90, CD73, ITGA6 (integrin alpha-6), CD146 and TM4SF1 (transmembrane 4 L6 family member 1).

Also, the stem cell of the present disclosure is negative for CD45 or CD34, specifically both CD45 and CD34.

In a specific exemplary embodiment of the present disclosure, the composition of the present disclosure decreases the activity or expression level of β-galactosidase (β-Gal). In the present specification, the term “decrease of activity or expression level” refers to the decrease of the expression level or intrinsic function of β-Gal in vivo such that cellular aging induced by SA-β-Gal can be inhibited or delayed to a detectable level. The decrease of activity includes not only the simple decrease of function but also the ultimate inhibition of activity caused by decreased stability. It may refer to a state where the activity or expression level has decreased specifically by 20% or more, more specifically by 40% or more, further more specifically by 60% or more, as compared to a control group.

In a specific exemplary embodiment of the present disclosure, the composition of the present disclosure induces collagen synthesis. In an example of the present disclosure, it was confirmed that, when the composition of the present disclosure is administered to a skin site where wound has been induced, the wound site is filled quickly as collagen synthesis is increased remarkably.

In a specific exemplary embodiment of the present disclosure, the composition of the present disclosure reduces reactive oxygen species (ROS) in cells. In an example of the present disclosure, it was observed that the amount of ROS accumulated in the cells treated with the composition of the present disclosure was less than 50% as compared to an untreated control group, confirming that the composition of the present disclosure can effectively protect cells, tissues and a subject from oxidative stress.

The present disclosure also provides a method for preventing oxidation or improving skin condition, which includes a step of administering the cosmetic composition of the present disclosure described above.

When the composition of the present disclosure is prepared as a cosmetic composition, it may contain an ingredient commonly used in a cosmetic composition, e.g., a common adjuvant such as a stabilizer, a solubilizer, a vitamin, a pigment and a fragrance, and a carrier in addition to the extracellular vesicle as an active ingredient.

The cosmetic composition of the present disclosure may be prepared into any formulation commonly prepared in the art. For example, it may be formulated into a solution, a suspension, an emulsion, a paste, a gel, a cream, a lotion, a powder, a soap, a surfactant-containing cleanser, an oil, a powder foundation, an emulsion foundation, a wax foundation, a spray, etc., although not being limited thereto. More specifically, it may be formulated into a softening lotion, a nourishing lotion, a nourishing cream, a massage cream, an essence, an eye cream, a cleansing cream, a cleansing foam, a cleansing water, a pack, a spray or a powder.

When the formulation of the present disclosure is a paste, a cream or a gel, an animal oil, a plant oil, a wax, paraffin, starch, tragacanth, a cellulose derivative, polyethylene glycol, silicone, bentonite, silica, talc, zinc oxide, etc. may be used as a carrier ingredient.

When the formulation of the present disclosure is a powder or a spray, lactose, talc, silica, aluminum hydroxide, calcium silicate or polyamide powder may be used as a carrier ingredient. In particular, a spray may further contain a propellant such as chlorofluorohydrocarbon, propane/butane or dimethyl ether.

When the formulation of the present disclosure is a solution or an emulsion, a solvent, a solubilizer or an emulsifier may be used as a carrier ingredient. Examples include water, ethanol, isopropanol, ethyl carbonate, ethyl acetate, benzyl alcohol, benzyl benzoate, propylene glycol, 1,3-butylene glycol, an aliphatic ester of glycerol, polyethylene glycol or an aliphatic ester of sorbitan.

When the formulation of the present disclosure is a suspension, a liquid diluent such as water, ethanol or propylene glycol, a suspending gent such as ethoxylated isostearyl alcohol, polyoxyethylene sorbitol ester and polyethylene sorbitan ester, microcrystalline cellulose, aluminum metahydroxide, bentonite, agar, tragacanth, etc. may be used as a carrier ingredient.

In a specific exemplary embodiment of the present disclosure, the stem cells of the present disclosure are obtained by:

(a) a step of treating an isolated human skeletal muscle tissue with a collagenase and then culturing the same;

(b) a step of centrifuging the culture of the step (a), and collecting and suspension culturing pellets; and

(c) a step of seeding the cells in the suspension culture of the step (b) on a culture dish.

According to the present disclosure, the skeletal muscle-derived stem cells of the present disclosure are isolated from skeletal muscle tissue, specifically orbicularis oculi muscle tissue, isolated from the human body by enzymatic digestion. Before treating with the collagenase, the isolated tissue may be cut into a regular size after removing blood and blood vessels. Then, after collecting pellets including stem cells through centrifugation and suspension culturing the same, the stem cells were seeded on a culture dish and then selected while observing the adhesion and morphology of the cells. The culture dish may be a gelatin-coated culture dish.

More specifically, the collagenase may be type II collagenase.

In a specific exemplary embodiment of the present disclosure, the extracellular vesicles of the present disclosure are obtained by:

(a) a step of culturing the skeletal muscle-derived stem cells;

(b) a step of collecting the culture of the step (a) and removing the stem cells through centrifugation;

(c) a step of filtering a supernatant obtained through the centrifugation with a vacuum filter; and

(d) a step of ultrafiltering the filtrate.

More specifically, the vacuum filter has a cut-off value of 0.15-0.3 μm. Further more specifically, it has a cut-off value of 0.18-0.25 μm. Most specifically, it has a cut-off value of 0.22 μm.

In the present specification, the term “ultrafiltration” refers to membrane-based separation by which the components constituting a non-homogeneous mixture solution are separated through a semipermeable membrane according to pressure or concentration gradient. An ultrafiltration membrane has pores with a predetermined cut-off value. In a specific exemplary embodiment of the present disclosure, the ultrafiltration is performed using a membrane having a cut-off value of 5-15 kDa, more specifically 10 kDa.

In the present disclosure, the vacuum filtration and the ultrafiltration may be performed either in order or in reverse order.

In another aspect, the present disclosure provides a pharmaceutical composition for preventing or treating a hyperpigmentation disease, which contains extracellular vesicles isolated from skeletal muscle-derived stem cells as active ingredients.

In another aspect, the present disclosure provides a method for preventing or treating a hyperpigmentation disease, which includes a step of administering the composition described above to a subject.

The detailed description of the skeletal muscle-derived stem cells and the extracellular vesicles isolated therefrom used in the present disclosure will be omitted to avoid redundancy because they were described in detail above.

In the present specification, the term “prevention” refers to suppression of the onset of a disease or a disorder in a subject with the risk of the disease or disorder although the subject has not been diagnosed as having the disease or disorder.

In the present specification, the term “treatment” refers to: (a) suppression of the progress of a disease, a disorder or a symptom; (b) amelioration of a disease, a disorder or a symptom; or (c) removal of a disease, a disorder or a symptom. When the composition of the present disclosure is administered to a subject, it suppresses, removes or ameliorates of the progress of a symptom related with hyperpigmentation by inhibiting melanocyte proliferation, tyrosinase activity and melanogenesis. Accordingly, the composition of the present disclosure may be used as a composition for treating a disease on its own, or may be administered as a therapeutic adjuvant for the disease together with another pharmacological ingredient. Therefore, in the present specification, the term “treatment” or “therapeutic agent” includes the meaning of “therapeutic support” or “therapeutic adjuvant”.

Because the composition of the present disclosure alleviates or reversibly restores skin pigmentation, the term “pharmaceutical composition for preventing or treating a hyperpigmentation disease” used in the present specification has the same meaning as “a pharmaceutical composition for skin whitening”.

In another aspect, the present disclosure provides a pharmaceutical composition for healing skin wound or reducing scar, which contains extracellular vesicles isolated from skeletal muscle-derived stem cells as active ingredients.

In another aspect, the present disclosure provides a method for healing skin wound or reducing scar, which includes a step of administering the composition described above to a subject.

In another aspect, the present disclosure provides a pharmaceutical composition for inhibiting skin aging, which contains extracellular vesicles isolated from skeletal muscle-derived stem cells as active ingredients.

In another aspect, the present disclosure provides a method for suppressing skin aging, which includes a step of administering the composition described above to a subject.

In the present specification, the term “administration” or “administer” refers to administration of a therapeutically effective amount of the composition of the present disclosure directly to a subject such that the same amount is formed in the body of the subject.

In the present disclosure, the term “therapeutically effective amount” refers to an amount of the pharmaceutical composition of the present disclosure wherein the content of the pharmacological ingredient (e.g., exosomes) in the composition is sufficient to provide therapeutic or prophylactic effect for a subject to which the composition is to be administered, and thus includes the meaning of “prophylactically effective amount”.

In the present specification, the term “subject” includes human, mouse, rat, guinea pig, dog, cat, horse, cow, pig, monkey, chimpanzee, baboon or rhesus monkey without limitation. Specifically, the subject of the present disclosure is human.

When the composition of the present disclosure is prepared as a pharmaceutical composition, the pharmaceutical composition of the present disclosure contains a pharmaceutically acceptable carrier.

The pharmaceutically acceptable carrier contained in the pharmaceutical composition of the present disclosure includes one commonly used for preparation, such as lactose, dextrose, sucrose, sorbitol, mannitol, starch, acacia gum, calcium phosphate, alginate, gelatin, calcium silicate, microcrystalline cellulose, polyvinylpyrrolidone, cellulose, water, syrup, methyl cellulose, methyl hydroxybenzoate, propyl hydroxybenzoate, talc, magnesium stearate, mineral oil, etc., although not being limited thereto. The pharmaceutical composition of the present disclosure may further contain a lubricant, a wetting agent, a sweetener, a flavorant, an emulsifier, a suspending agent, a preservative, etc. in addition to the above-described ingredients. Suitable pharmaceutically acceptable carriers and preparations are described in detail in Remington's Pharmaceutical Sciences (19th ed., 1995).

The pharmaceutical composition of the present disclosure may be administered orally or parenterally. Specifically, it may be administered parenterally, more specifically subcutaneously or transdermally.

An appropriate administration dosage of the pharmaceutical composition of the present disclosure may be determined variously depending on such factors as preparation method, mode of administration, the age, body weight, sex, pathological condition or diet of a patient, administration time, administration route, excretion rate and response sensitivity. A preferred administration dosage of the pharmaceutical composition of the present disclosure is within a range of 0.001 to 100 mg/kg for an adult.

The pharmaceutical composition of the present disclosure may be prepared as a single-dose or multiple-dose forms using a pharmaceutically acceptable carrier and/or excipient according to a method that can be easily carried out by those having ordinary knowledge in the art to which the present disclosure belongs. It may be formulated as a solution in an oily or aqueous medium, a suspension, a syrup, an emulsion, an extract, a dust, a powder, a granule, a tablet or a capsule, and may further contain a dispersant or a stabilizer.

### Advantageous Effects

The features and advantages of the present disclosure may be summarized as follows:

(a) The present disclosure provides a composition for improving skin condition having the effect of skin aging delay, wound healing, scar reduction and skin whitening, which contains extracellular vesicles isolated from skeletal muscle-derived stem cells as active ingredients.

(b) The present disclosure can not only utilize tissues wasted after surgical operation, thus allowing easy supply of raw materials, but also takes advantage of natural substances, thereby being free from the risk of side effects even upon long-term administration.

(c) The composition of the present disclosure promotes the secretion of collagen, reduces reactive oxygen species and promotes re-epithelialization at wound site while remarkably inhibiting melanocyte proliferation, tyrosinase activity and melanogenesis. Accordingly, it can facilitate effective recovery from skin tissue damage caused by oxidative stress, physical wound, intrinsic aging at cell or tissue levels, or hyperpigmentation.

### BRIEF DESCRIPTION OF DRAWINGS

FIGS. 1a-1e show orbicularis oculi muscle-derived mesenchymal stem cells acquired from the skeletal muscle of a patient. FIG. 1a schematically shows a process of isolating ORM-SCs from the tissue of a patient. First, the skeletal muscle tissue of a patient is incised with a laser blade and then digested enzymatically (stirred at 37° C. for 1 hour and 30 minutes with type II collagenase). After centrifugation, pellets are collected and incubated on a 0.2% gelatin-coated culture plate. FIG. 1b shows the morphology of ORM-SCs depending on sex and age (F: female, M: male, numbers: age, scale bar: 50 μm). FIG. 1c shows eyelid surgery. FIG. 1d shows a result of measuring the doubling time of orbicularis oculi muscle-derived stem cells. FIG. 1e shows a result of measuring the cumulative cell number of orbicularis oculi muscle-derived stem cells.

FIGS. 2a-2f show the characteristics of ORM-SCs. FIG. 2a shows a result of analyzing the colony-forming unit of ORM-SCs, and FIG. 2b shows the mRNA expression level of stemness markers (Nanog, Sox2 and Rex1) in WJ-MSCs and ORM-SCs. FIG. 2c shows a result of analyzing the mRNA expression level of surface markers for distinction between ORM-SCs and HDFs. FIG. 2d shows that ORM-SCs differentiate into adipocytes, chondrocytes and osteocytes for 14-21 days. Adipocyte differentiation was measured with oil red O staining, chondrocyte differentiation with alcian blue staining, and osteocyte differentiation with alizarin red S staining. FIG. 2e shows an RT-PCR result showing the expression of CD markers. Positive and negative CD markers in ORM-SCs were compared with those in WJ-MSCs, and the markers were investigated by flow cytometry.

FIGS. 3a-3e show a result of analyzing the isolation of ORM-SC-EVs. FIG. 3a schematically shows a process of isolating ORM-SC-EVs. A process of removing cells, dead cells and cell debris from a conditioned medium through differential centrifugation and collecting ORM-SC-EVs through ultrafiltration is summarized. FIG. 3b shows an immunoblotting result showing the expression of CD63, CD81, calnexin and GM130 in ORM-SC-EVs. FIG. 3c shows a result of observing the vesicular morphology of ORM-SC-EVs by transmission electron microscopy (TEM) (scale bar=100 nm). FIG. 3d shows a result of analyzing the size distribution of ORM-SC-EVs through dynamic light scattering (DLS). FIG. 3e shows the concentration (particles/mL) of ORM-SC-EVs measured through nanoparticle tracking analysis (NTA).

FIGS. 4a-4e show the inhibition of melanin synthesis by ORM-SC-EVs. FIG. 4a shows a result of analyzing cell viability at maximum ORM-SC-EV concentration using CCK-8. No significant difference in cell viability was observed at concentrations from 2 to 50 μg/mL, but the proliferation of B16F10 cells was decreased significantly at 100 μg/mL. *versus vehicle, **p<0.01. FIG. 4b shows a result of treating melanoma cell with α-MSH (200 nM) for 24 hours and then treating with ORM-SC-EVs (5, 10, 30 or 50 μg/mL) for 48 hours for investigation of intracellular melanin. Arbutin (100 μM) was used as a positive control group. *versus α-MSH alone, *p<0.05, ****p<0.0001. FIG. 4c shows a result of conducting experiment under a similar condition for investigation of extracellular melanin. *versus α-MSH alone, *p<0.05. **p<0.01, ***p<0.001, ****p<0.0001. FIGS. 4d and 4e show a result of, after treating melanoma cells with α-MSH (200 nM), incubating the cells with ORM-SC-EVs (5, 10, 30 or 50 μg/mL) or arbutin (100 μM) as a positive control group and measuring tyrosinase activity (FIG. 4d) and expression level (FIG. 4e) with the same time intervals. *versus α-MSH alone, **p<0.01, ****p<0.0001.

FIG. 5a schematically shows the inhibition of melanogenesis mediated by ORM-SC-EVs. It shows that EVs are obtained effectively from the stem cells isolated from the tissue wasted after eyelid surgery.

FIG. 5b schematically summarizes an experimental procedure for investigating the melanin-regulating effect of ORM-SC-EVs using B16F10 cells.

FIGS. 6a-6c show melanogenesis in B16F10 cells treated with ORM-SC-EVs. FIG. 6a shows cell morphology when the cells were treated with ORM-SC-EVs at different concentrations (5, 10, 30 or 50 μg/mL). Melanosomes are indicated with yellow arrows. FIG. 6b shows significant change in color after melanin synthesis (α-MSH group; black, ORM-SC-EVs 50 μg/mL group; gray). FIG. 6c shows a result of measuring tyrosinase activity using L-DOP.

FIGS. 7a-7c show a cell experiment result showing the antiaging and antioxidant activity of ORM-SC-EVs. They show the result of investigating the change in the activity of β-galactosidase (SA-β-Gal) by ORM-SC-EVs (FIG. 7a), the change in the intracellular accumulation of H2DCFDA (FIG. 7b) and the change in the expression of antioxidation-related genes (FIG. 7c).

FIGS. 8a-8f show the wound healing and skin regeneration effects of ORM-SC-EVs. They show the result of in-vitro and in-vivo wound healing assays (FIGS. 8a, 8b, 8d and 8e), the change in the expression level of skin regeneration- and scar reduction-related factors (FIG. 8c) and the effect on re-epithelialization and regeneration at wound site investigated through analysis of collagen synthesis (FIG. 8f).

FIG. 9 schematically shows a process of obtaining ORM-SC-EVs of the present disclosure and their effects.

### BEST MODE

Hereinafter, the present disclosure is described in more detail through examples. The following examples are provided only to illustrate the present disclosure more specifically, and it will be obvious to those having ordinary knowledge in the art that the scope of the present disclosure is not limited by the examples.

### Examples

### Experimental Methods

Isolation of ORM-SCs from Human Tissue

Human ORM tissue (diameter 1 cm, weight 0.5 g) was acquired from Konkuk University Hospital. ORM-SCs were isolated by enzymatic digestion. Briefly, the tissue was washed twice with PBS and blood and blood vessels were removed to prevent contamination. Then, the tissue was incised with a knife and incubated with type II collagenase at 37° C. for 1 hour while stirring for 30 minutes with 5-minute intervals. After the incubation, the digested tissue was centrifuged twice at 1,500 rpm for 10 minutes to remove the remaining collagenase. Then, after suspending the tissue pellets in an α-MEM (Gibco) medium containing 10% FBS (fetal bovine serum; Peak Serum) and 1% penicillin/streptomycin (Gibco) and then seeding on a 0.2% gelatin-coated culture dish, the adhesion and morphology of the cells were observed with a microscope while incubating at 37° C. and 5% CO₂.

### Colony-Forming Unit of ORM-SCs

Colony-forming unit was measured to investigate the self-renewal ability of the ORM-SCs. 1×10³ ORM-SCs were seeded on a 6-well plate and cultured at 37° C. and 5% CO₂ for 12 days. After the culturing, the cells were washed and stained with 0.15% crystal violet. After washing again with PBS, the colony was imaged.

### RT-PCR

After lysing the ORM-SCs with a Labozol reagent (LaboPass, CMRZ001), total RNA was isolated according to the manufacturer's instructions. The purified RNA was quantified using a NanoDrop spectrophotometer (ND-ONE). cDNA synthesis was performed using a M-MuLV reverse transcription kit (Labopass, CMRT010) and oligo dT primers. PCR was conducted using a rTaq Plus 5×PCR master mix (ELPIS Biotech, EBT-1319) and the PCR product was visualized using 1-2% agarose gel. The primer sequences described in Tables 1-3 were used.

TABLE 1
Primer sequences of stem cell CD markers
used in RT-PCR

| | Forward primer | Reverse primer |
|---|---|---|
| | (5′ to 3′) | (5′ to 3′) |
| CD19 | CCTGGGGTCCCAGTCCTATG | GCTCCAGAGGTTGGCATCAT |
| CD45 | TCAGTGGTCCCATTGTTGTG | GCATCTCTGTGGCCTTAGCT |
| CD29 | GCCGCGCGGAAAAGATG | ACATCGTGCAGAAGTAGGCA |
| CD73 | TATCCGGTCGCCCATTGATG | ACGCTATGCTCAAAGGCCTT |
| CD105 | CCAAGACCGGGTCTCAAGAC | TGTACCAGAGTGCAGCAGTG |
| CD166 | GAACACGATGAGGCAGACGA | CCGAGGTCCTTGTTTACATG |
| | | TTT |
| GAPDH | AATCCCATCACCATCTTCCA | ATGACCCTTTTGGCTCCC |
| | G | |

TABLE 2
Primer sequences of stem cell sternness
markers used in RT-PCR

| | Forward primer | Reverse primer |
|---|---|---|
| | (5′ to 3′) | (5′ to 3′) |
| NANOG | TCCTGAACCTCAGCTACAAAC | GCGTCACACCATTGCTATTC |
| SOX2 | CATCACCCACAGCAAATGAC | GAAGTCCAGGATCTCTCTCA |
| | | TAAA |
| REX1 | GTTTCGTGTGTCCCTTTCAAG | CTGTTATCTGCTTCATCCTG |
| | | TTG |
| GAPDH | AATCCCATCACCATCTTCCAG | ATGACCCTTTTGGCTCCC |

TABLE 3
Primer sequences of stem cell surface
markers used in RT-PCR

| | Forward primer | Reverse primer |
|---|---|---|
| | (5′ to 3′) | (5′ to 3′) |
| ITGA6 | CGAAACCAAGGTTCTG | CTTGGATCTCCACTGA |
| | AGCCCA | GGCAGT |
| CD146 | GTGTTGAATCTGTCTT | ATGCCTCAGATCGATG |
| | GTGAA | |
| TM4SF1 | GGCTACTGTGTCATTG | ACTCGGACCATGTGGA |
| | TGGCAG | GGTATC |
| GAPDH | AATCCCATCACCATCT | CACGATACCAAAGTTG |
| | TCCAG | TCATG |

### Flow Cytometry

Immunophenotyping assay of the ORM-SCs was conducted by flow cytometry. Briefly, the cells were trypsinized to obtain a single cell suspension and then reacted with primary and secondary antibodies on ice for 10 minutes. The primary antibodies used in the present disclosure were CD34 (R&D Systems, MAB72271), CD45 PD7/26/16+2B11 (Invitrogen, MA5-13197), CD73/NT5E (Invitrogen, RG235718), CD90/Thy1 (R&D Systems, AF2067) and CD105 (Invitrogen, MA5-11854). The intensity of fluorescence emitted from the labeled antibodies was measured with a flow cytometer (BD Bioscience, San Jose, Calif. USA).

Differentiation into Three Lineages

The ORM-SCs were induced to differentiate into fat, bone and cartilage for 2 weeks. For differentiation into fat, the ORM-SCs were exposed to an adipocyte differentiation medium containing 10% low-glucose DMEM supplemented with 5 μg/mL insulin, 500 μM isobutylmethylxanthine (IBMX) and 1 μM dexamethasone. An osteocyte differentiation medium contained 10% low-glucose DMEM supplemented with 50 μg/mL L-ascorbic acid, 10 nM β-glycerophosphate and 100 nM dexamethasone, and a chondrocyte differentiation medium contained 10% low-glucose DMEM, 100 nM dexamethasone, 10 nM β-glycerophosphate, 50 μg/mL L-ascorbic acid, 10 μg/mL TGF-β3, 1 mM sodium pyruvate, 40 μg/mL proline and 1× insulin transferrin-selenium.

The differentiation into bone, fat and cartilage could be confirmed by staining with alizarin red S, oil red and alcian blue, respectively.

### Isolation of ORM-SC-EVs

For isolation of EVs, 4×10⁶ ORM-SCs were seeded on a 150-mm cell culture dish together with serum-free α-MEM. The culture was collected and centrifuged differentially at 300 g for 10 minutes to remove the cells. Then, the supernatant was transferred cautiously to a fresh tube and the cell debris was removed by centrifuging at 2000 g for 10 minutes. Then, the supernatant was transferred again to a fresh tube and centrifuged at 2000 g for 1 hour. The obtained supernatant was filtered through a 0.22-μm vacuum filter (EMD Millipore SCGP00525 Steriflip-GP filter) to remove vesicles. EVs were separated from the filtrate by performing ultrafiltration using an Equilibrate Amicon® Ultra-15 filter (#UFC901024, 10 kDa MWCO). Finally, the EVs were concentrated by centrifuging at 4,000 g for 30 minutes.

### Characterization of ORM-SC-EVs

Proteins in the isolated EVs were quantitated using a BCA protein assay kit (Pierce, Waltham, Mass., USA) according to the manufacturer's protocol. The size of the EVs was investigated by dynamic light scattering (DLS) using Nano Zetasizer (Malvern Instruments, Malvern, UK). The number of the EVs was measured using the nanoparticle tracking analyzer NS300 (Nanosight, Amesbery, UK).

The morphology and structure of the EVs were analyzed at 80 kV using a transmission electron microscope (TEM, JEM-1010, Nippon Denshi, Tokyo, Japan). The ORM-SC-EVs and ORM-SC lysate were separated by 4-12% SDS-PAGE and transferred onto a PVDF (polyvinylidene difluoride) membrane (ThermoFisher, 1624001). The membrane was blocked using 5% skim milk. After incubation overnight at 4° C. with anti-CD63 (Invitrogen, 10628D), anti-CD81 (Santa Cruz, sc-7637), anti-calnexin (CST, 2679T), anti-GM130 (CST, 12480S) and anti-β-actin (CST, 4970S) antibodies, followed by reaction with a secondary antibody (HRP; horseradish protein) at room temperature, protein signals were detected using an enhanced chemiluminescence kit (Amersham Biosciences, USA) and a ChemiDoc™ imaging system (Bio-Rad, 17001401).

### Cell Proliferation Assay

For analysis of cell proliferation after exposure to the EVs, B16F10 cells were seeded on a 96-well plate at 3×10³ cells/well and retained in a serum-free RPMI medium for 24 hours. Then, the cells were exposed to the ORM-SC-EVs at different concentrations (2, 5, 10, 30, 50 and 100 μg/mL) for 48 hours. After the incubation, 10 μL of a CCK-8 solution (Dojindo, CK04-05) was added to each well and light was blocked after incubation for 2 hours. Then, absorbance was measured at 450 nm using a Bio-Rad x-Mark™ spectrophotometer (Bio-Rad Laboratories, USA).

### Measurement of Melanin Content

B16F10 cells were retained on a 12-well plate at 4×10⁴ cells/well using an RPMI medium containing 10% FBS. After culturing for 24 hours, the cells were treated with 200 nM α-melanocyte-stimulating hormone (α-MSH) (Sigma, M4135) and the ORM-SC-EVs (5, 10, 30, 50 μg/mL) or arbutin (100 μM) (Sigma, A4256) and then cultured for 60 hours. For measurement of extracellular melanin content, 100 μL of the culture medium was transferred to a fresh 96-well plate and absorbance was measured at 405 nm using a Bio-Rad x-Mark™ spectrophotometer. For measurement of intracellular melanin content, each well was washed with PBS and the cells were lysed at 80° C. for 1 hour with 200 μL of 1 N NaOH. Then, absorbance was measured at 405 nm. The extracellular and intracellular melanin contents were normalized to total proteins.

### Tyrosinase Activity Assay

B16F10 cells were cultured for 24 hours on a 12-well plate at a density of 4×10⁴ cells/well. Then, after treating each well with 200 nM α-MSH (Sigma, M4135) and the ORM-SC-EVs (5, 10, 30 or 50 μg/mL) or arbutin (100 μM) (Sigma, A4256), the cells were cultured for 48 hours. After washing each well with PBS, the cells were separated with PBS and suspended in a 50 mM phosphate buffer (pH 6.8) containing 1% Triton X-100. After vortexing, the mixture was incubated at −80° C. for 30 minutes and then thawed at room temperature. After centrifuging at 1000 g for 10 minutes, 40 μL of the supernatant and 100 μL of 10 mM L-DOPA (Sigma, 333786) were added to a 96-well plate. After incubation at 37° C. for 1 hour, absorbance was measured at 405 nm using a Bio-Rad x-Mark™ spectrophotometer. The absorbance value was normalized to total proteins.

### β-Galactosidase (SA-β-Gal) Activity Assay

For investigation of the antioxidant activity of the orbicularis oculi muscle stem cell-derived extracellular vesicles (OOM-SC-EV) on cellular level, normal human dermal fibroblasts (NHDF, PromoCell, c-23020) were subcultured for 13-15 passages to induce aging. Then, after treating with the OOM-SC-EVs, the cells were stained with senescence-associated β-galactosidase (SA-β-gal) according to a previously reported method (Nature Protocols, 2009. 4(12): p. 1798). Briefly, the cells were seeded on a 4-well cell culture plate (SPL, 30004) and treated with the OOM-SC-EVs at a concentration of 50 μg/mL 12 hours later. After removing the culture, followed by addition of 1 mL of 1×PBS (Veratech) and washing twice at 100 rpm for 5 minutes, the cells were fixed for 15 minutes by adding 1 mL of 2% paraformaldehyde and 0.2% glutaraldehyde. Then, after discarding the fixative solution and adding 1 mL of 1×PBS, the cells were washed twice at 100 rpm for 5 minutes. After adding 1 mL of a previously prepared SA-β-gal staining solution, incubation was conducted for 5 hours at 37° C. under a CO₂-free condition. Then, after discarding the SA-β-gal staining solution and adding 1 mL of 1×PBS, the cells were washed twice at 100 rpm for 5 minutes. After adding 1 mL of 100% MeOH (Samjin Industrial), the cells were left alone at room temperature for 30 minutes. Then, after discarding the 100% MeOH and adding 1×PBS, the cells were observed under an optical microscope (Fusion 100, Chemyx). The composition of the SA-β-gal staining solution was as follows: 200 mM citric acid/phosphoric acid, 100 mM K₄[Fe(CN)₆].3H₂O, 100 mM K₃[Fe(CN)₆], 5 M NaCl, 1 M MgCl₂, 50 mg/mL X-gal. The cells positive for SA-β-gal are stained blue.

### Evaluation of Reactive Oxygen Species Production (H2DCFDA)

A 2′,7′-dichlorodihydrofluorescein diacetate (H2DCFDA, Invitrogen) reagent was used for measurement of intracellular accumulation of reactive oxygen species (ROS). NHDFs were treated with the OOM-SC-EVs (50 μg/mL) and incubated at 37° C. in high-glucose DMEM (Sigma, D6429; 1% P/S, 10% FBS). After discarding the culture medium, the cells were washed cleanly 1-2 times with 1×PBS and high-glucose DMEM (1% P/S, w/o FBS) was added. After adding H2DCFDA to a final concentration of 10 μM, the cells were incubated at 37° C. and 5% CO₂ for 30 minutes. After discarding the culture medium containing 10 μM H2DCFDA, the NHDFs were washed 1-2 times with 1×PBS and then observed using a fluorescence microscope (Nikon Eclipse TE2000-E).

### Measurement of Expression of Antioxidation-Related Genes

The change in the mRNA expression level of antioxidation-related genes in NHDFs treated with the OOM-SC-EVs (50 μg/mL) was investigated by performing real-time PCR. NHDFs were treated with the OOM-SC-EV at 50 μg/mL. About 24 hours later, the cells were lysed with a Labozol reagent (LaboPass, CMRZ001) and total RNA was isolated according to the manufacturer's instructions. The purified RNA was quantitated using a NanoDrop spectrophotometer (ND-ONE). cDNA synthesis was conducted using a M-MuLV reverse transcription kit (Labopass, CMRT010) and oligo dT primers. Real-time PCR (Amersham Phamacia Biotech 7500) was conducted using a HiPi real-time PCR 2× master mix (SYBR Green, ROX, 500rxn) (ELPIS Biotech, EBT-1802). The primer sequences described in Table 4 were used.

TABLE 4
Primer sequences used in real-time PCR of
antioxidation-related genes

| | Forward primer | Reverse primer |
|---|---|---|
| Gene | (5′ to 3′) | (5′ to 3′) |
| GPX1 | CAGTCGGTGTATGCCTTCT | GAGGGACGCCACATTCTCG |
| | CG | |
| GPX2 | GGTAGATTTCAATACGTTC | TGACAGTTCTCCTGATGTC |
| | CGGG | CAAA |
| GPX3 | AGAGCCGGGGACAAGAGAA | ATTTGCCAGCATACTGCTT |
| | | GA |
| GPX4 | GAGGCAAGACCGAAGTAAA | CCGAACTGGTTACACGGGA |
| | CTAC | A |
| GPX7 | CCCACCACTTTAACGTGCT | GGCAAAGCTCTCAATCTCC |
| | C | TT |
| GSR | TTCCAGAATACCAACGTCA | GTTTTCGGCCAGCAGCTAT |
| | AAGG | TG |
| SOD1 | GGTGGGCCAAAGGATGAAG | CCACAAGCCAAACGACTTC |
| | AG | C |
| SOD2 | GCTCCGGTTTTGGGGTATC | GCGTTGATGTGAGGTTCCA |
| | TG | G |
| SOD3 | ATGCTGGCGCTACTGTGTT | CTCCGCCGAGTCAGAGTTG |
| | C | |
| Catalase | TGTTGCTGGAGAATCGGGT | TCCCAGTTACCATCTTCTG |
| | TC | TGTA |
| TMX1 | AGTATGTCAGCACTCTTTC | CACACTGGCAATCCAAGGT |
| | AGC | CT |
| TXNIP | GGTCTTTAACGACCCTGAA | ACACGAGTAACTTCACACA |
| | AAGG | CCT |
| TXN | GTGAAGCAGATCGAGAGCA | CGTGGCTGAGAAGTCAACT |
| | AG | ACTA |

### In-Vitro Wound Healing Assay

For evaluation of the effect of the OOM-SC-EVs on the migration of NHDFs, NHDFs were cultured on a 6-well cell culture plate (SPL, 30006) to 95% confluency and the proliferation was stopped by treating with 10 μg/mL mitomycin C (Sigma, M4287) for 2 hours. After washing with PBS, the culture plate was scratched using a 200 μL tip end and then treated with the OOM-SC-EVs at 5 μg/mL and 50 μg/mL. The cells were observed with a microscope with 12-hour intervals.

### Comparison of Skin Regeneration-, Wrinkle Improvement- and Scar Reduction-Associated Factors

Real-time PCR was conducted to investigate the change in the mRNA expression level of skin regeneration- and wrinkle improvement-associated genes in NHDFs treated with the OOM-SC-EVs (50 μg/mL). The expression of type 1 collagen (Col1A1), monocyte chemoattractant protein (MCP)-1, MCP-3, chemokine ligand 5 (CCL-5), plasminogen activator inhibitor-1 (PAI-1), TFG-β₁ (transforming growth factor beta 1) and TGF-β₃ (transforming growth factor beta 3) in NHDFs treated with the OOM-SC-EVs (50 μg/mL) was measured using the primers described in Table 5.

TABLE 5
Primer sequences used in real-time PCR of
skin regeneration-, wrinkle-improvement
and scar reduction-related genes

| | Forward primer | Reverse primer |
|---|---|---|
| | (5′ to 3′) | (5′ to 3′) |
| Col1A1 | GATTCCCTGGACCTAAAGG | AGCCTCTCCATCTTTGCCA |
| | TGC | GCA |
| MCP-1 | AGAATCACCAGCAGCAAGT | TCCTGAACCCACTTCTGCT |
| | GTCC | TGG |
| MCP-3 | ACAGAAGGACCACCAGTAG | GGTGCTTCATAAAGTCCTG |
| | CCA | GACC |
| CCL5 | CCTGCTGCTTTGCCTACAT | ACACACTTGGCGGTTCTTT |
| | TGC | CGG |
| PAI-1 | CTCATCAGCCACTGGAAAG | GACTCGTGAAGTCAGCCTG |
| | GCA | AAAC |
| TGF-β₁ | TACCTGAACCCGTGTTGCT | GTTGCTGAGGTATCGCCAG |
| | CTC | GAA |
| TGF-β₃ | CTAAGCGGAATGAGCAGAG | TCTCAACAGCCACTCACGC |
| | GATC | ACA |

### In-Vivo Wound Healing Assay

6-week-old female BALB/c nude mice were used for experiments after accustomation for a week. After anesthesia, full-thickness wound was formed on the back using an 8-mm biopsy punch (Kai, BP-80F). Then, the OOM-SC-EVs (50 μg/mL) were injected into the wound site over three times. PBS was injected to a control group. Silicone (0.5 mmT) and Tegaderm (1622W) tapes punched with an 8-mm biopsy punch were used to protect the wound site, and wound site was imaged at regular time intervals using an 8-mm punched silicone tape.

### Histological Analysis

On day 14 after the induction of wound, tissue including the whole wound was taken and fixed for 48 hours in a 4% paraformaldehyde solution. Tissue sections passing through the center of the wound were taken, dehydrated and then embedded in paraffin blocks. After sectioning the tissue with a microtome, H&E (hematoxylin & eosin) staining was conducted after removing paraffin by attaching to a polylysine-coated slide and hydrating the tissue. Meanwhile, the slide was placed in a Weigert's iron hematoxylin solution for 10 minutes and in Biebrich scarlet-acid fuchsin and aniline blue for 5 minutes, respectively, for Masson's trichrome staining.

### Statistical Analysis

All statistical analysis was performed using GraphPad Prism (version 7). Most experiments were repeated in triplicate. P-value was calculated by one-way ANOVA and t-test using GraphPad Prism and then statistical significance was evaluated. In all the figures, *p<0.05. **p<0.01, ***p<0.001 and ****p<0.0001.

### Experimental Results

Isolation of ORM-SCs from Patients' Tissues

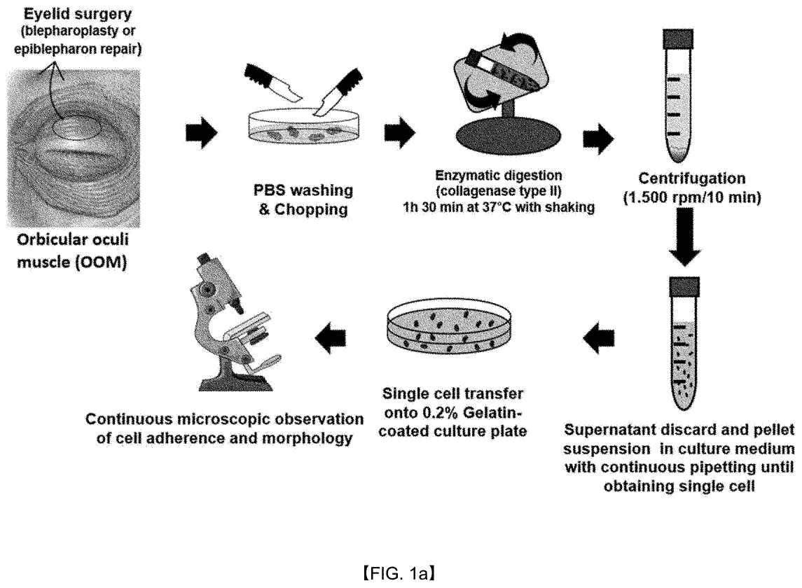

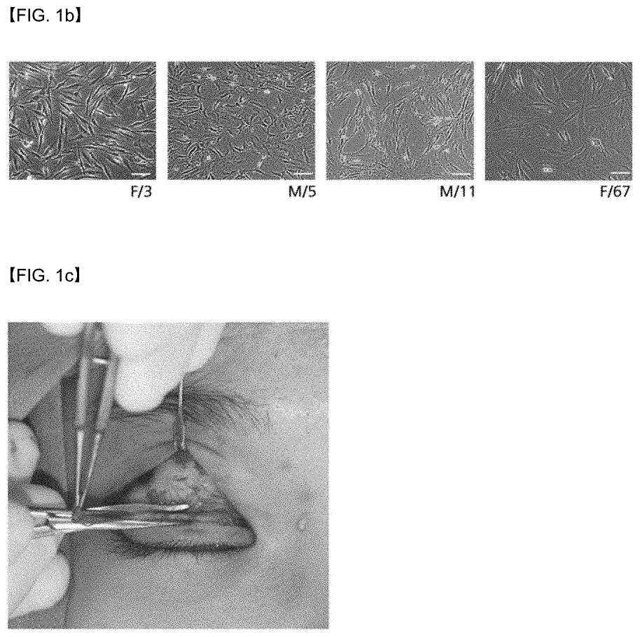

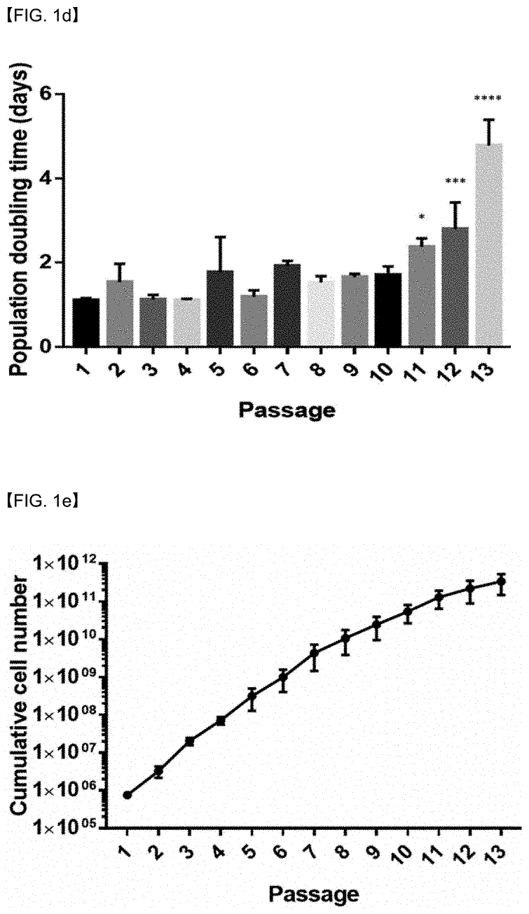

The ORM is composed of the orbital, septal and tarsal (eyelid) portions [14]. The skeletal muscle samples used in the present disclosure were taken from the eyelids of patients who received eyelid surgery. The patients' tissue samples were obtained through the same surgical procedure [15] and the sex and age of the patients were recorded. After incising the ORM with a laser blade and digesting enzymatically, centrifugation was performed at 1,500 rpm for 10 minutes. Then, cell pellets were seeded on a 0.2% gelatin-coated culture plate (FIG. 1a). No significant difference was observed for the morphology of ORM-SCs depending on sex and age (n=4) (FIG. 1b).

### Population Doubling Time of Orbicularis Oculi Muscle-Derived Stem Cells

Population doubling time (PDT) was measured while subculturing the orbicularis oculi muscle-derived stem cells for 13 passages (P13). It was confirmed that PDT was maintained almost constant for 24 hours until passage 10 (P10), but was increased slightly from passage 11 and remarkably from passage 13 (FIG. 1d).

### Measurement of Cumulative Cell Number of Orbicularis Oculi Muscle-Derived Stem Cells

Cumulative cell number was measured while subculturing the orbicularis oculi muscle-derived stem cells. After calculating an expansion factor by dividing the number of harvested cells by the number of seeded cells, the cumulative cell number was calculated by multiplying the calculated expansion factor by the number of cells harvested in the previous passage. As a result of measuring the cumulative cell number while culturing for 3 days per passage on average, the increase in the cumulative cell number was maintained constant until passage 10 and the increasing rate was decreased from passage 11 (FIG. 1e).

### Characterization of Differentiation Ability and Sternness of ORM-SCs

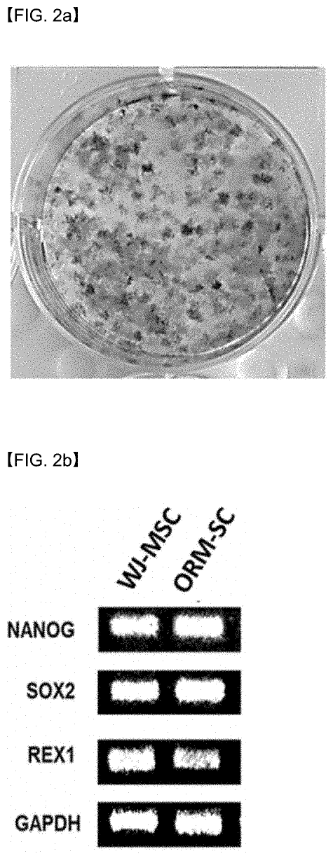

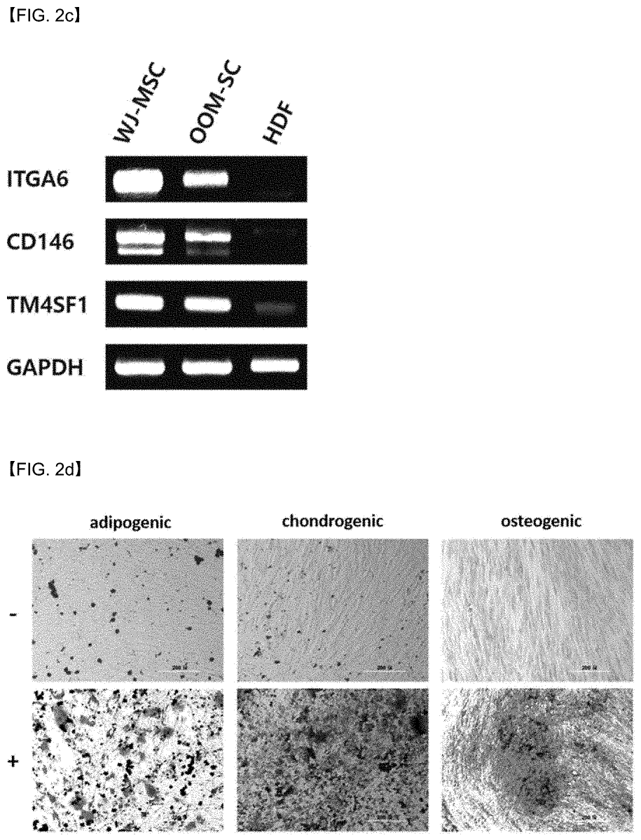

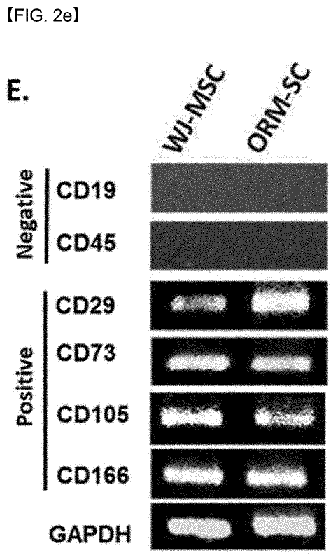

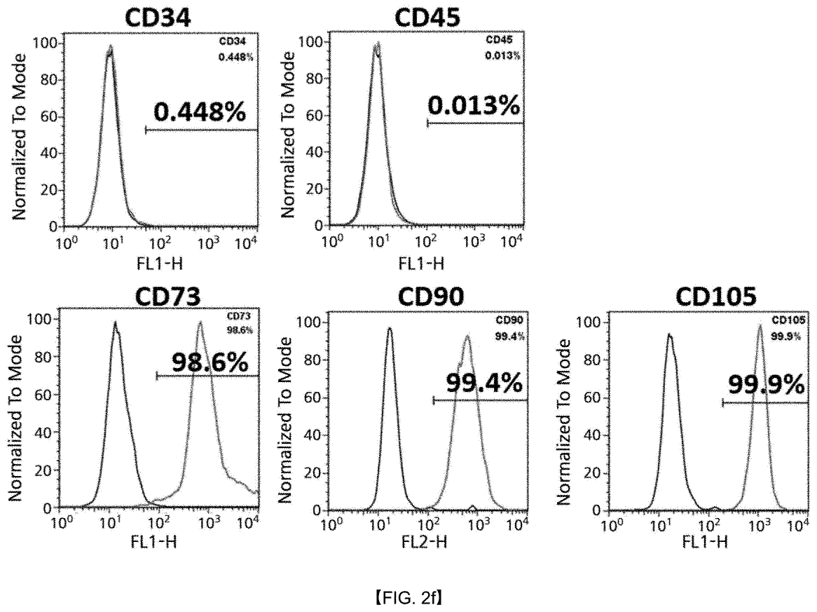

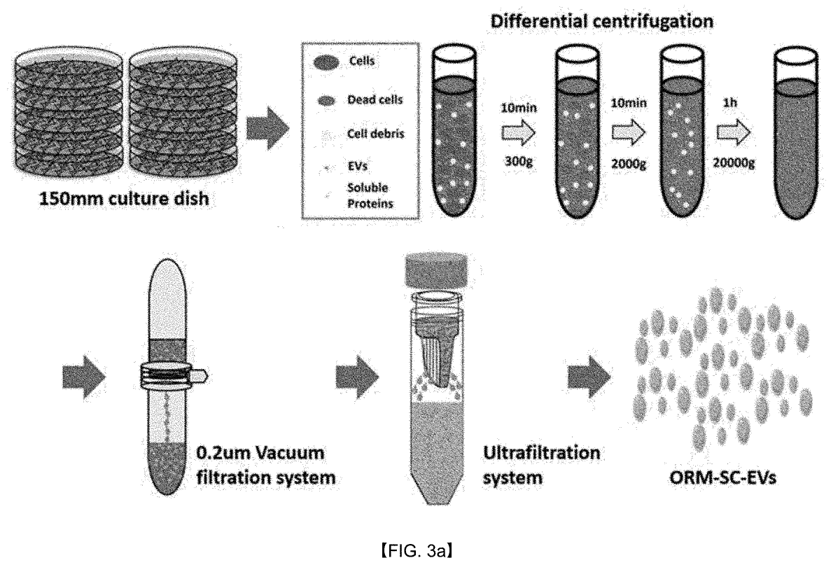

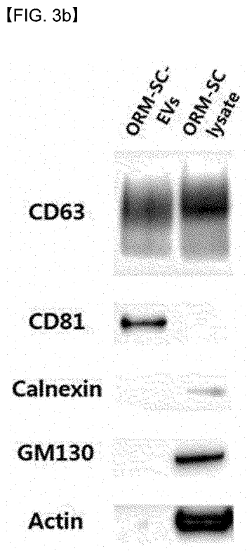

The isolated ORM-SCs were spindle-shaped. The ORM-SCs the were cultured for 12 days in vitro grew fast and showed high colony-forming ability (FIGS. 1b and 2a). For investigation of the expression of stemness markers in the ORM-SCs, the expression level of Nanog, Sox2 and Rex1 was measured by RT-PCR and was compared with that of WJ-MSCs (FIG. 2b). The multipotency (adipocyte, chondrocyte and osteocyte differentiation) of the ORM-SCs cultured in a differentiation induction medium was investigated by staining with oil red O, alizarin red S and alcian blue (FIG. 2d). In addition, the mRNA expression of CD markers was investigated for characterization of mesenchymal stem cells. As a result of RT-PCR, positive CD markers were highly expressed in the ORM-SCs as compared to the WJ-MSCs, whereas negative CD markers were not expressed (FIG. 2e). Through flow cytometry, it was observed that the positive mesenchymal stem cell surface markers (CD105, CD90 and CD73) were highly expressed in the ORM-SCs but the negative markers (CD45 and CD34) were not expressed (FIG. 2f). In addition, ITGA6 (integrin alpha-6), CD146 (melanoma cell adhesion molecule) and TM4SF1 (transmembrane 4 L6 family member 1) were highly expressed in the WJ-MSCs and the ORM-SCs, but were not expressed in the HDFs (FIG. 2c).

Characterization of ORM-SC-EVs Isolated from ORM-SCs

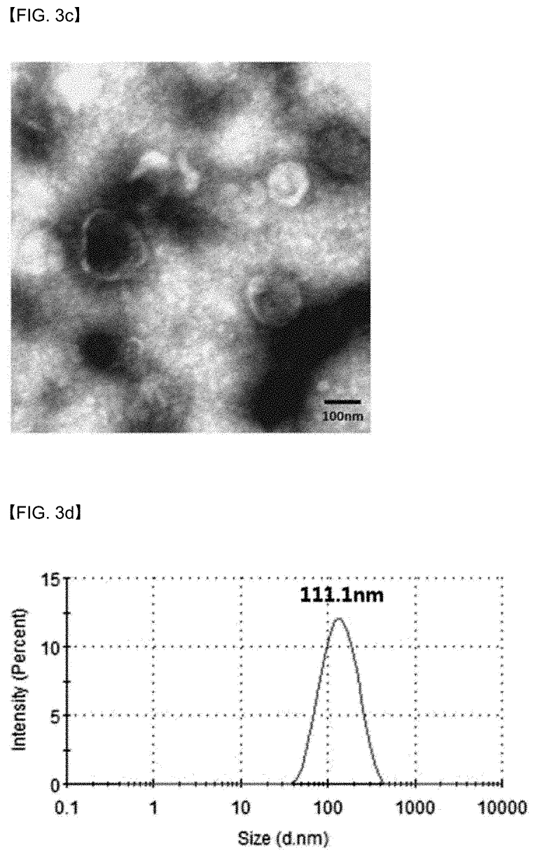

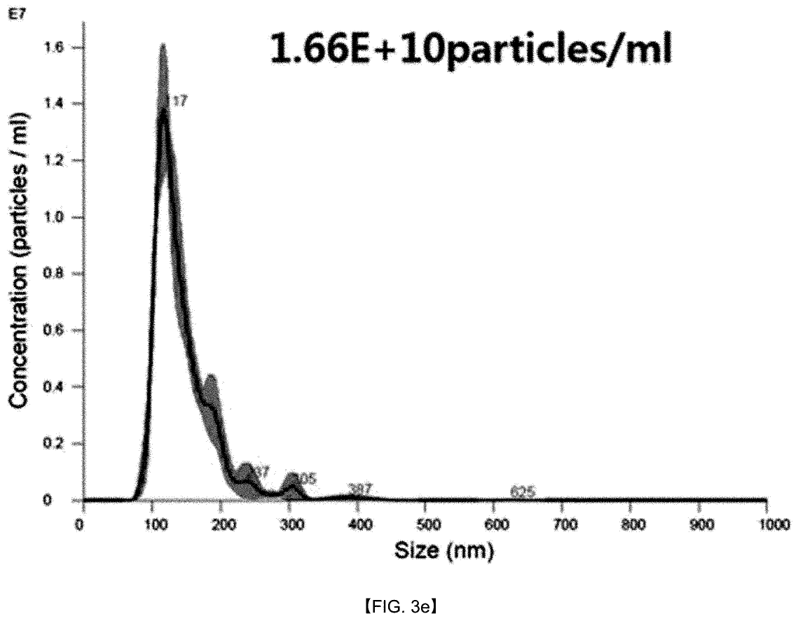

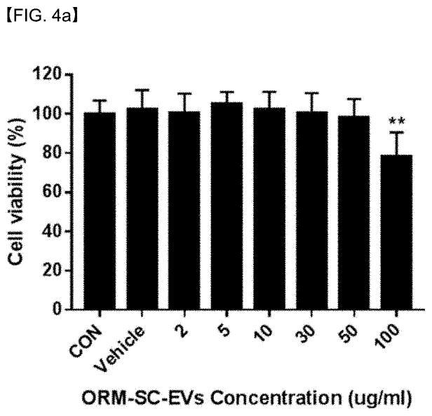

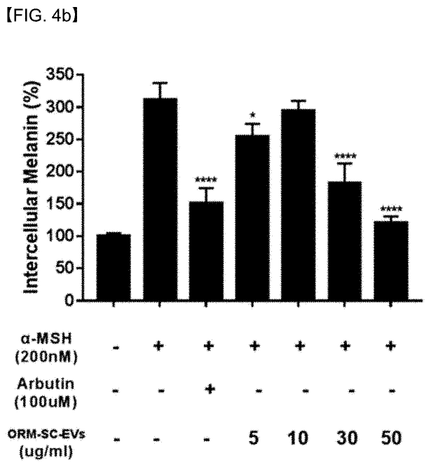

The yield of the EV particles was the highest for the Amicon 10-k Da (pore size) cellulose membrane as compared to other pore sizes or membranes. The ORM-SCs were cultured in a serum-free medium for 24 hours until 90% confluency. Differential centrifugation and ultrafiltration were performed to remove cells, dead cells and cell debris. Then, the ORM-SC-EVs were collected using a filtration system (FIG. 3a). For confirmation of EV-associated positive markers, the expression of CD63 and CD81 proteins was investigated by immunoblotting (FIG. 3b). As a result of western blotting, the expression of calnexin and GM130 proteins was not observed in the ORM-SC-EVs (FIG. 3b). The size distribution of the ORM-SC-EVs was investigated by dynamic light scattering using Nanosizer. The ORM-SC-EVs had an average diameter of 111.1 nm. The concentration of the ORM-SC-EVs measured by nanoparticle tracking analysis was 1.66×10¹⁰ particles/mL (FIGS. 3d and 3e). The ORM-SC-EVs were imaged with a transmission electron microscope. The ORM-SC-EVs were cup- or sphere-shaped (FIG. 3c).

### Melanin Synthesis Inhibition Effect of ORM-SC-EVs

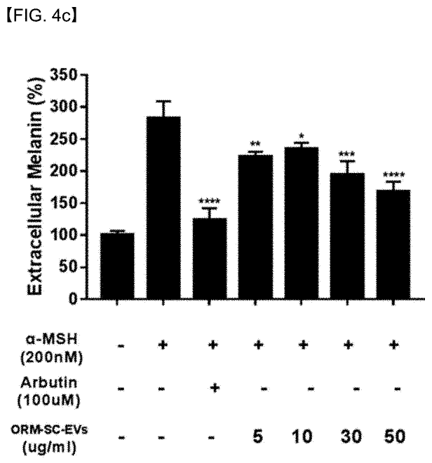

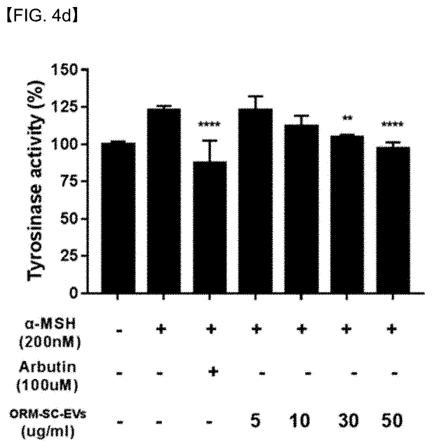

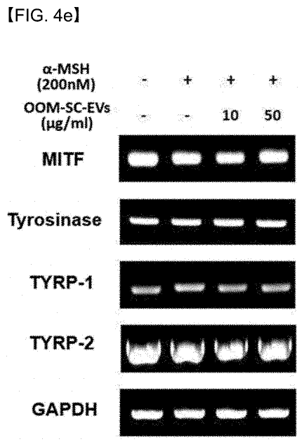

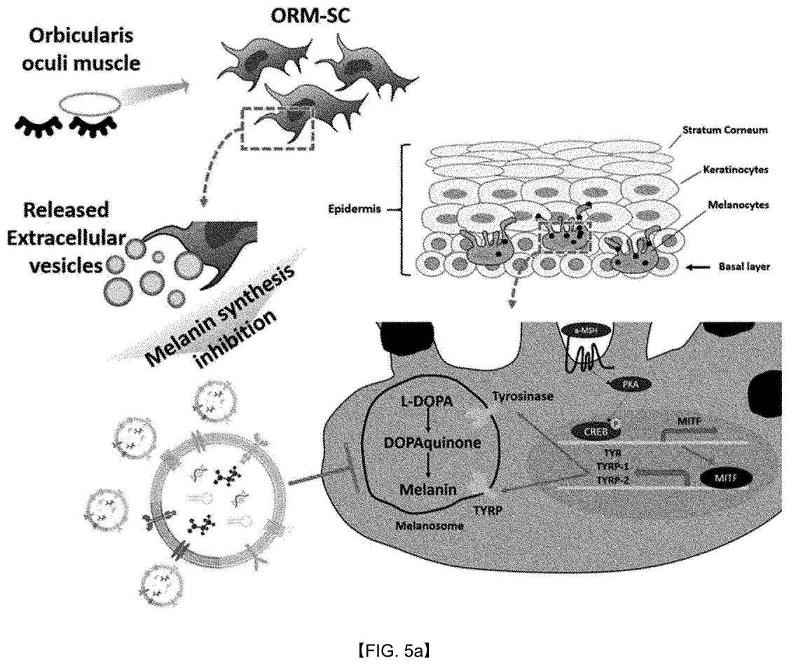

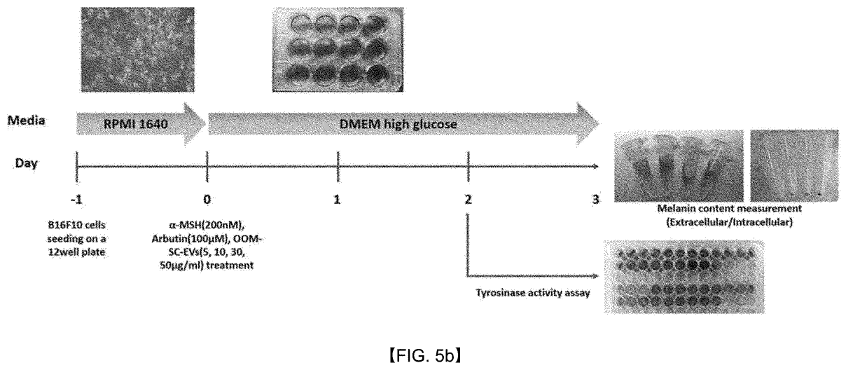

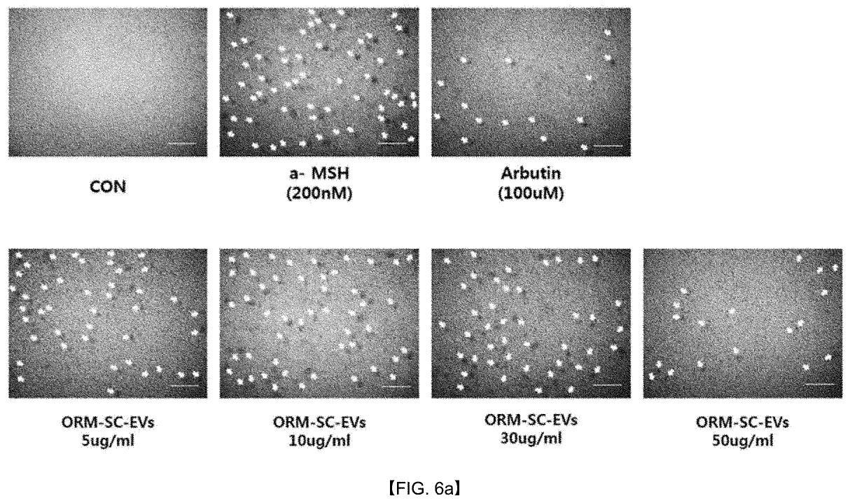

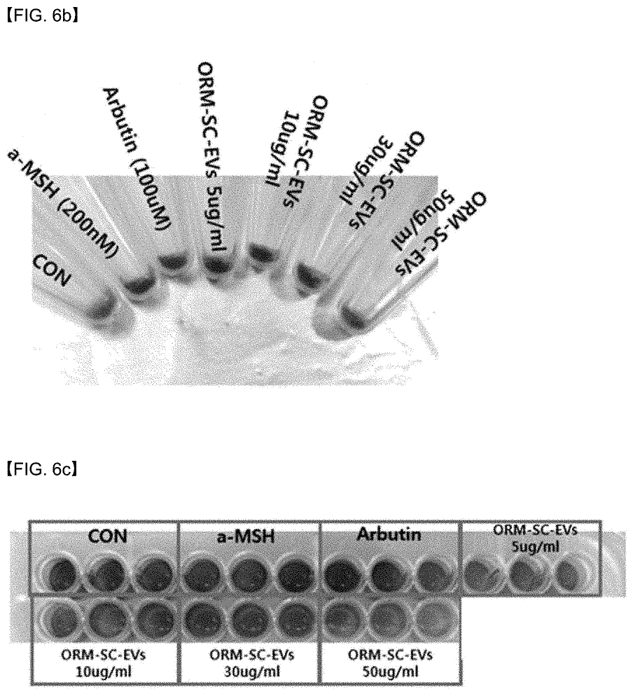

The melanin synthesis inhibition effect of the ORM-SC-EVs was investigated by measuring the viability of B16F10 melanoma cells depending on the concentration of ORM-SC-EVs using CCK-8. As a result, no effect was observed at 50 μg/mL and the viability of melanoma cells was decreased at 100 μg/mL (FIG. 4a). In another experiment, melanin synthesis was induced by treating melanoma cells with α-MSH (200 nM) and then melanin synthesis was inhibited by treating with the ORM-SC-EVs (5, 10, 30 or 50 μg/mL) and arbutin (100 μM). As a result of observing melanosome production, the secretion of melanosomes was decreased as the concentration of the ORM-SC-EVs was increased (FIG. 6a). Accordingly, it can be seen that both intercellular and extracellular melanin are decreased by the ORM-SC-EVs (FIGS. 4b and 4c). The decreased melanin synthesis was observed also when the cells were sedimented (FIG. 6b). As a result of measuring the activity and expression level of tyrosinase, which is an important factor in melanogenesis, significant difference was observed for the test group treated with 50 μg/mL ORM-SC-EVs (FIGS. 4d and 4e), and this result was also verified visually (FIG. 6c).

### SA-β-Gal Activity Assay

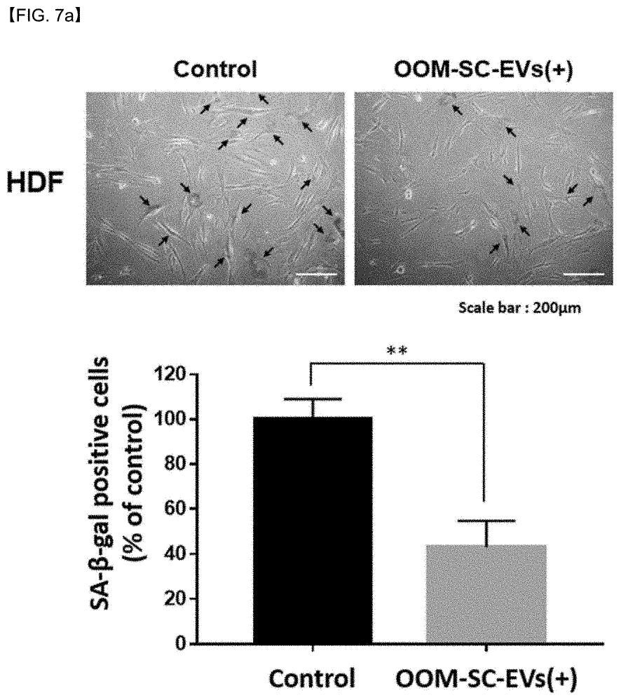

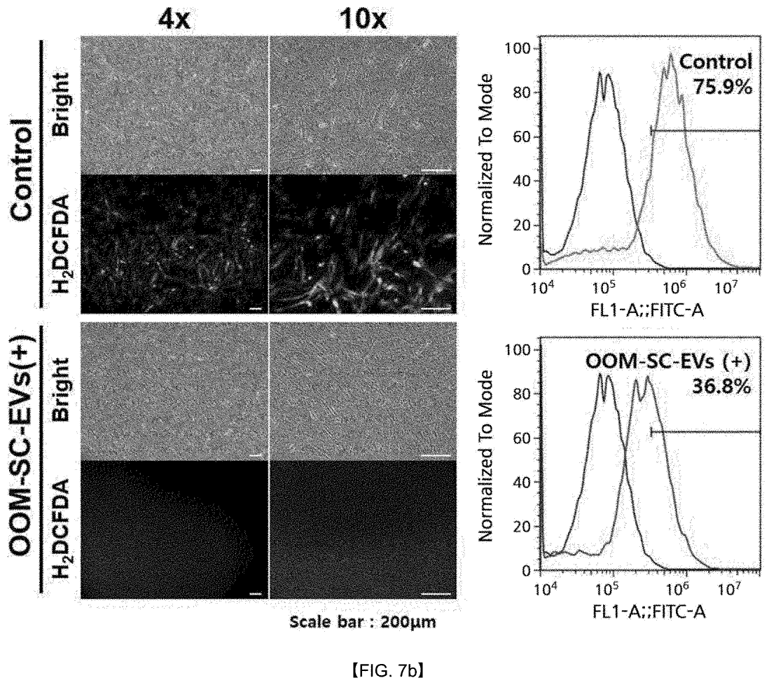

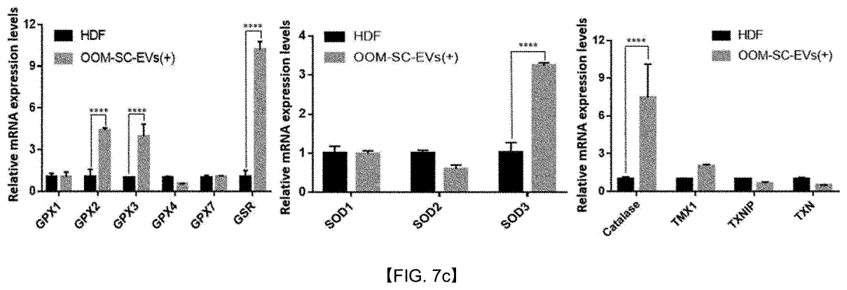

For investigation of the antiaging effect of the OOM-SC-EVs on cellular level, senescent NHDFs were stained with β-galactosidase. As a result, the β-galactosidase activity of the group treated with the OOM-SC-EVs was decreased to about 43% with respect to the cells of the control group as 100% (FIG. 7a). Through this, it was confirmed that the composition of the present disclosure can significantly inhibit the aging of human dermal fibroblasts.

### Assessment of Antioxidant Activity

For investigation of the change in the accumulation of intracellular reactive oxygen species (ROS) by the OOM-SC-EVs, the final concentration of H2DCFDA was measured with a flow cytometer (Beckman Coulter/CytoFLEX). As a result, it was confirmed that, the groups treated with the OOM-SC-EV showed 36.8% staining, whereas the control group showed about 75.9% staining (FIG. 7b). In addition, the expression of antioxidation-related genes was compared through real-time PCR. The group treated with the OOM-SC-EVs showed remarkable increase of the GPX2 (about 4 times), GPX3 (about 4 times), GSR (about 10 times), SOD3 (about 3 times) and catalase (about 8 times) genes as compared to the control group (FIG. 7c). Through this, it was confirmed that the composition of the present disclosure exhibits significant antioxidant activity.

### Wound Healing Assay

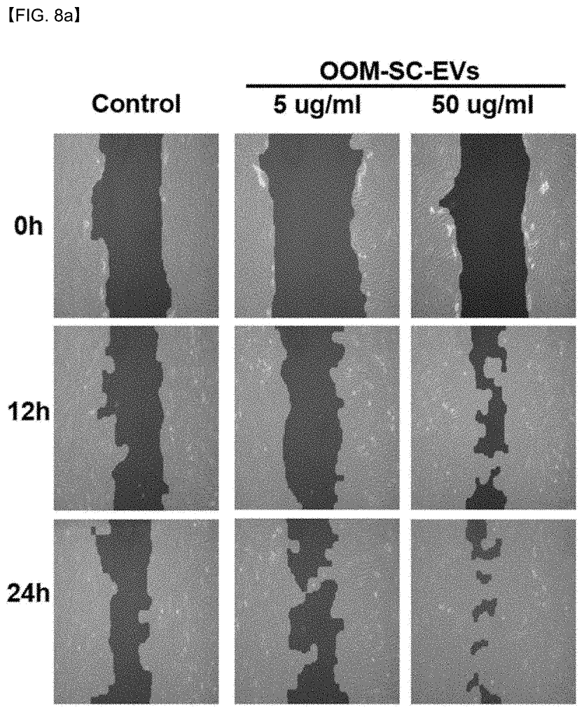

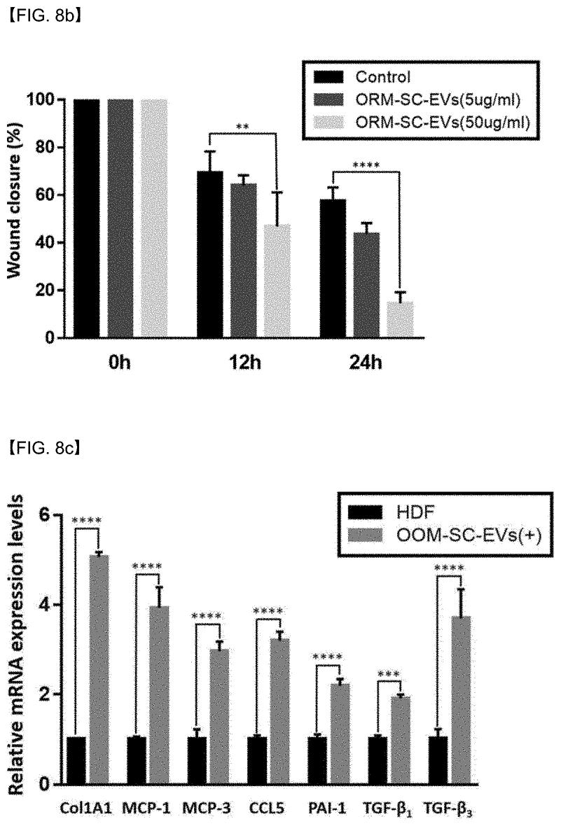

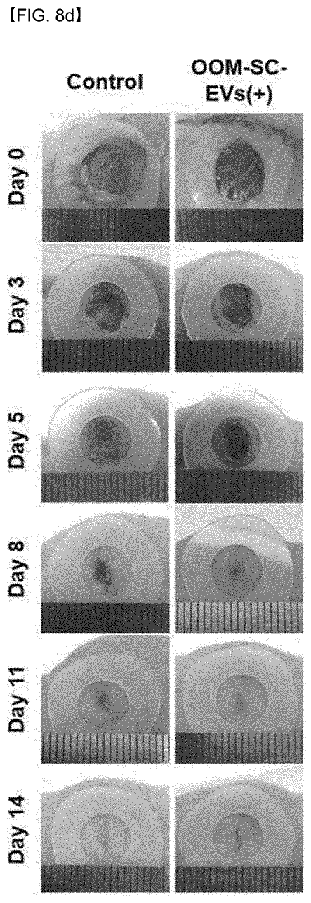

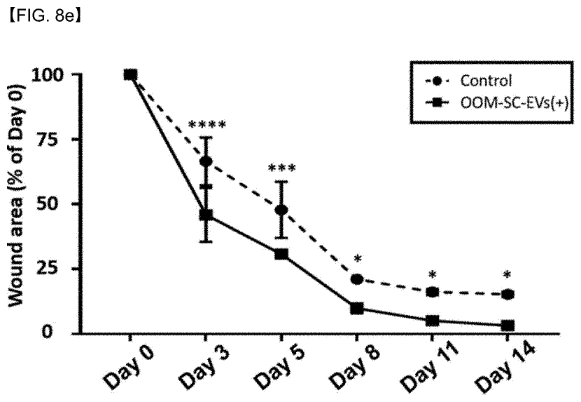

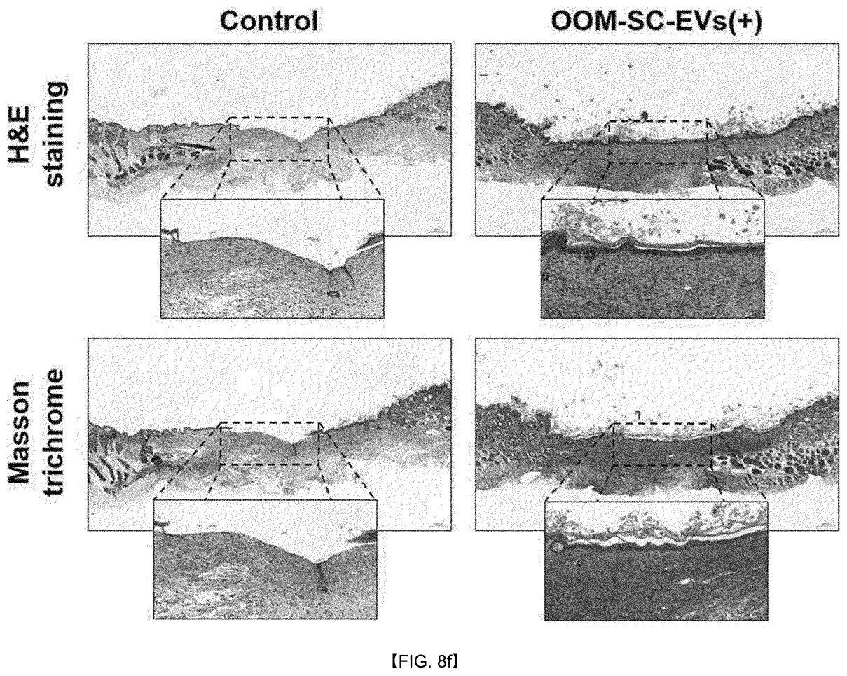

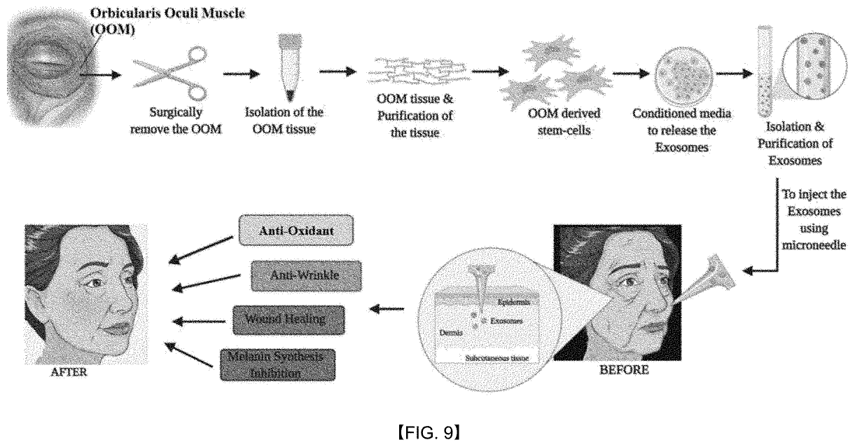

After stopping the proliferation of NHDFs with mitomycin C, followed by scratching, the cells were treated with the OOM-SC-EVs. As a result, the group treated with 50 μg/mL OOM-SC-EVs showed significantly improved migration ability as compared to the control group. That is to say, the wound formed by the scratching was filled significantly (FIGS. 8a and 8b).

In addition, the size of the wound sites formed on four mice with a biopsy punch was compared. As a result, it was confirmed that the area of the wound site was decreased significantly for the group treated with the OOM-SC-EVs. Accordingly, it can be seen that the composition of the present disclosure can effectively induce wound healing in vivo, too (FIGS. 8d and 8e).

In addition, histological analysis was performed on the wound-induced site through H&E staining and Masson's trichrome staining. As a result of the H&E staining, it was confirmed that the group treated with the OOM-SC-EVs showed faster re-epithelialization of the wound as compared to the control group. As a result of the Masson's trichrome staining, it was confirmed that more collagen was synthesized in the group treated with the OOM-SC-EVs (FIG. 8f).

### Analysis of Skin Regeneration, Wrinkle Improvement and Scar Reduction Effects

Real-time PCR was conducted to investigate the change in the mRNA expression level of skin regeneration- and wrinkle improvement-associated genes in the group treated with the OOM-SC-EVs (50 μg/mL). As a result, it was confirmed that the expression of type 1 collagen (Col1A1), which is involved in the facilitation of collagen synthesis and inhibition of collagen degradation, monocyte chemoattractant protein (MCP-1), MCP-3, chemokine ligand 5 (CCL-5) and plasminogen activator inhibitor-1 (PAI-1), which is associated with skin regeneration, was increased in the group treated with the OOM-SC-EVs (FIG. 8c). In addition, it was confirmed that the composition of the present disclosure significantly increases the ratio of the expression level of TGF-β₃ (transforming growth factor beta 3) to the expression level of TGF-β₁ (transforming growth factor beta 1), which are factors associated with inhibition of scar tissue formation or reduction of scar area [16,17] (FIG. 8c). Through this, it can be seen that the composition of the present disclosure can not only quickly regenerate and recover skin wound but also effectively remove, reduce or improve scar tissue formed together with discoloration of skin.

While the specific exemplary embodiments of the present disclosure have been described in detail, it will be obvious to those having ordinary knowledge in the art that the foregoing description is merely illustrative and the scope of the present disclosure is not limited thereby. Accordingly, it is to be understood that the substantial scope of the present disclosure is defined by the appended claims and their equivalents.

### REFERENCES

1. Dominici, M., et al., Cytotherapy, 2006. 8(4): p. 315-317.
2. Via, A. G., A. Frizziero, and F. Oliva, Muscles, ligaments and tendons journal, 2012. 2(3): p. 154-162.
3. Gao, X., et al., Biomaterials, 2014. 35(25): p. 6859-6870.
4. Levy, M. M., et al., Bone, 2001. 29(4): p. 317-322.
5. Sun, J.-S. et al., Biomaterials, 2005. 26(18): p. 3953-3960.
6. Scuderi, N. and C. Rubino, British Journal of Plastic Surgery, 1994. 47(1): p. 57-59.
7. Zhou, G., Clinical Experience with Orbicularis Oculi Myocutaneous Flaps in the Temporal Area. Plastic and reconstructive surgery, 2003. 112(7): p. 1862.
8. Yoshimura, Y., T. Nakajima, and K. Yoneda, Plastic and reconstructive surgery, 1991. 88(1): p. 136-139.
9. Kostakoglu, N. and G. OEzcan, Burns, 1999. 25(6): p. 553-557.
10. Sekulic-Jablanovic, M., et al., The Journal of General Physiology, 2016. 147(5): p. 395.
11. Teixeira, F. G., et al., Cellular and Molecular Life Sciences, 2013. 70(20): p. 3871-3882.
12. Keshtkar, S., N. Azarpira, and M. H. Ghahremani, Stem Cell Research & Therapy, 2018. 9(1): p. 63.
13. Ando, H., et al., Journal of Investigative Dermatology, 2012. 132(4): p. 1222-1229.
14. Liu, G., et al., Tissue Engineering and Regenerative Medicine, 2018. 15(4): p. 445-452.
15. Costin, B. R., et al., Ophthalmic Plastic & Reconstructive Surgery, 2015. 31(4).
16. Namazi, M. R., et al., International journal of dermatology, 2011. 50(1), 85-93.
17. Wang, L., Hu, L., et al., Scientific Reports, 2017. 7(1), 1-12.

We claim:

1. A cosmetic composition for antioxidation or improvement of skin condition, comprising extracellular vesicles isolated from skeletal muscle-derived stem cells as active ingredients, wherein the improvement of skin condition is selected from a group consisting of skin aging delay, wound healing, scar reduction and skin whitening.

2. The composition according to claim 1, wherein the skeletal muscle is orbicularis oculi muscle.

3. The composition according to claim 1, wherein the extracellular vesicles have a diameter of 50-200 nm.

4. The composition according to claim 1, wherein the extracellular vesicles are comprised in the composition at a concentration of 5-100 μg/mL.

5. The composition according to claim 1, wherein the stem cells are positive for CD105, CD90, CD73, ITGA6 (integrin alpha-6), CD146 and TM4SF1 (transmembrane 4 L6 family member 1), and negative for CD45 and CD34.

6. The composition according to claim 1, wherein the composition decreases the activity or expression level of β-galactosidase (β-Gal).

7. The composition according to claim 1, wherein the composition induces collagen synthesis.

8. The composition according to claim 1, wherein the composition reduces reactive oxygen species (ROS) in cells.

9. The composition according to claim 1, wherein the stem cells are obtained by:
(a) a step of treating an isolated human skeletal muscle tissue with a collagenase and then culturing the same;
(b) a step of centrifuging the culture of the step (a), and collecting and suspension culturing pellets; and
(c) a step of seeding the cells in the suspension culture of the step (b) on a culture dish.

10. The composition according to claim 9, wherein the collagenase is type II collagenase.

11. The composition according to claim 1, wherein the extracellular vesicles are obtained by:
(a) a step of culturing the skeletal muscle-derived stem cells;
(b) a step of collecting the culture of the step (a) and removing the stem cells through centrifugation;
(c) a step of filtering a supernatant obtained through the centrifugation with a vacuum filter; and
(d) a step of ultrafiltering the filtrate.

12. The composition according to claim 11, wherein the vacuum filter has a cut-off value of 0.15-0.3 μm.

13. The composition according to claim 11, wherein the ultrafiltration is performed using a membrane having a cut-off value of 5-15 kDa.

14. A pharmaceutical composition for preventing or treating a hyperpigmentation disease, comprising extracellular vesicles isolated from skeletal muscle-derived stem cells as active ingredients.

15. A pharmaceutical composition for healing skin wound or reducing scar, comprising extracellular vesicles isolated from skeletal muscle-derived stem cells as active ingredients.

16. A pharmaceutical composition for inhibiting skin aging, comprising extracellular vesicles isolated from skeletal muscle-derived stem cells as active ingredients.

17. The composition according to claim 14, wherein the skeletal muscle is orbicularis oculi muscle.
